(19) United States
(12) Patent Application Publication
(10) Pub. No.: US 2004/0063133 A1
(43) Pub. Date: Apr. 1, 2004

(54) Method of normalizing gene expression data

(75) Inventors: Masato Some, Kanagawa-ken (JP); Nobuhiko Ogura, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD

(21) Appl. No.: 10/671,546

(22) Filed: Sep. 29, 2003

(30) Foreign Application Priority Data
Sep. 30, 2002 (JP) 285102/2002

(51) Int. Cl. C12Q 1/68; G06F 19/00; G01N 33/48; G01N 33/50

(52) U.S. Cl. 435/6; 702/20

(57) ABSTRACT

Data concerning gene expression quantities, which have been obtained for first and second samples, are indicated with points plotted on a logarithmic coordinate system, in which a horizontal axis represents logarithms of the expression quantities obtained for the first sample, and in which a vertical axis represents logarithms of the expression quantities obtained for the second sample. A coefficient is calculated from a value of an intercept of an approximate straight line, which is obtained from approximate representation of the plotted points with a straight line having a slope of 1, on the vertical axis. The data concerning the expression quantities with respect to a plurality of genes, which expression quantities have been obtained for the second sample, are divided by the coefficient and are thus normalized.

### BACKGROUND OF THE INVENTION

1. Field of the Invention

This invention relates to a method of normalizing gene expression data wherein, in a process for comparing gene expression data with respect to one of two samples, which gene expression data have been obtained from hybridization with a plurality of genes, and gene expression data with respect to the other sample, which gene expression data have been obtained from the hybridization with the plurality of the genes, with each other, the gene expression data with respect to either one of the samples are normalized.

2. Description of the Related Art

Genetic information within an organism is stored as a DNA base sequence, and an analysis of gene expression is efficient for prevention of various diseases, early diagnosis and treatment of various diseases, made-to-order medical treatment of various diseases, and the like. For gene analyses in the fields of biology and medical science, a micro array technique has heretofore been utilized as a technique for analyzing gene expression. With the micro array technique, a DNA chip or a DNA micro array, which comprises a slide glass and several thousands of DNA spots formed on the slide glass, is prepared, and a sample is subjected to hybridization with the DNA spots. Also, expression quantities of genes are determined with intensities of hybrid formation being taken as indexes.

Recently, a technique for monitoring gene expression by the utilization of the micro array technique has been developed. Ordinarily, a state of a disease is characterized by a difference in expression levels of various genes due to alteration of copy number of a genetic DNA of a specific gene or alteration of a transfer level. For example, deletion or acquisition of a genetic material plays an important role in cancer growth or cancer progress. Also, alteration of the expression level of a specific gene acts as an index for the presence and progress of various cancers. Therefore, in order for an analysis of gene expression to be made, it is necessary that expression levels of a plurality of genes in a diseased cell and the expression levels of the plurality of the genes in a normal cell be compared with each other.

Since, for example, the amount of a gene extracted from a sample varies for experiments, in cases where a quantitative analysis of gene expression is to be made by the utilization of the micro array technique, gene expression data have heretofore been normalized by use of a measured value acting as a reference value. Heretofore, a process for comparing expression quantities with respect to a plurality of genes, which expression quantities have been obtained for one of two samples, and the expression quantities with respect to the plurality of the genes, which expression quantities have been obtained for the other sample, with each other has been performed in the manner described below. Specifically, a certain gene, which is expressed certainly from both the samples is located as a reference probe on a micro array. Also, it is assumed that the expression quantity with respect to the certain gene, which expression quantity is obtained for one of the two samples, and the expression quantity with respect to the certain gene, which expression quantity is obtained for the other sample, should be identical with each other. On the assumption described above, the gene expression data, which have been obtained for either one of the samples, are normalized such that the expression quantity with respect to the certain gene, which expression quantity has been obtained for one of the two samples, and the expression quantity with respect to the certain gene, which expression quantity has been obtained for the other sample, become identical with each other.

Further, a different process for comparing expression quantities with respect to a plurality of genes, which expression quantities have been obtained for one of two samples, and the expression quantities with respect to the plurality of the genes, which expression quantities have been obtained for the other sample, with each other has heretofore been performed in the manner described below. Specifically, it is assumed that the total sum of the expression quantities with respect to all of the genes, which expression quantities are obtained for one of two samples, and the total sum of the expression quantities with respect to all of the genes, which expression quantities are obtained for the other sample, will be identical with each other. On the assumption described above, the gene expression data, which have been obtained for either one of the samples, are normalized such that the total sum of the expression quantities with respect to all of the genes, which expression quantities have been obtained for one of the two samples, and the total sum of the expression quantities with respect to all of the genes, which expression quantities have been obtained for the other sample, become identical with each other.

However, with the normalizing process utilizing the reference probe described above, the fundamental problems described below occur. Specifically, the expression quantity with respect to the certain gene, which is expressed certainly from both the samples, is not necessarily representative of the expression quantities with respect to all of the genes. Therefore, in cases where the gene expression data, which have been obtained for either one of the samples, are normalized such that the expression quantity with respect to the certain gene, which expression quantity has been obtained for one of the two samples, and the expression quantity with respect to the certain gene, which expression quantity has been obtained for the other sample, become identical with each other, the expression quantities with respect to the other genes do not become equal to predetermined values. Accordingly, with the normalizing process utilizing the reference probe described above, the accuracy of the analysis cannot be kept high.

Also, with the aforesaid normalizing process, wherein it is assumed that the total sum of the expression quantities with respect to all of the genes, which expression quantities are obtained for one of two samples, and the total sum of the expression quantities with respect to all of the genes, which expression quantities are obtained for the other sample, will be identical with each other, the problems described below occur. Specifically, the data with respect to genes having large expression quantities become dominant, and the normalized gene expression data are largely affected by the data with respect to the genes having large expression quantities. Also, with the aforesaid normalizing process, wherein it is assumed that the total sum of the expression quantities with respect to all of the genes, which expression quantities are obtained for one of two samples, and the total sum of the expression quantities with respect to all of the genes, which expression quantities are obtained for the other sample, will be identical with each other, gene expression quantities of noise levels are also contained in the summation. However, the gene expression quantities are comparatively small. Therefore, the problems occur in that error cannot be kept small.

### SUMMARY OF THE INVENTION

The primary object of the present invention is to provide a method of normalizing gene expression data wherein, in a process for comparing expression quantities with respect to a plurality of genes, which expression quantities have been obtained for one of two samples, and expression quantities with respect to the plurality of the genes, which expression quantities have been obtained for the other sample, with each other, gene expression data with respect to either one of the samples are capable of being normalized appropriately.

The present invention provides a first method of normalizing gene expression data wherein, in a process for comparing expression quantities with respect to a plurality of genes, which expression quantities have been obtained for a first sample, and expression quantities with respect to the plurality of the genes, which expression quantities have been obtained for a second sample, with each other, data concerning the expression quantities having been obtained for the second sample are normalized, the method comprising the steps of:

i) indicating the data concerning the expression quantities, which have been obtained for the first sample and the second sample, with points plotted on a logarithmic coordinate system, in which a horizontal axis represents logarithms of the expression quantities obtained for the first sample, and in which a vertical axis represents logarithms of the expression quantities obtained for the second sample,

ii) calculating a coefficient from a value of an intercept of an approximate straight line, which is obtained from approximate representation of the plotted points with a straight line having a slope of 1, on the vertical axis, and

iii) performing division processing for dividing the data concerning the expression quantities with respect to the plurality of the genes, which expression quantities have been obtained for the second sample, by the coefficient, whereby the data concerning the expression quantities having been obtained for the second sample are normalized.

The present invention also provides a second method of normalizing gene expression data wherein, in a process for comparing expression quantities with respect to a plurality of genes, which expression quantities have been obtained for a first sample, and expression quantities with respect to the plurality of the genes, which expression quantities have been obtained for a second sample, with each other, data concerning the expression quantities having been obtained for the second sample are normalized, the method comprising the steps of:

i) indicating the data concerning the expression quantities, which have been obtained for the first sample and the second sample, with points plotted on a coordinate system, in which a horizontal axis represents the expression quantities obtained for the first sample, and in which a vertical axis represents the expression quantities obtained for the second sample,

ii) calculating a value of a slope of an approximate straight line, which is obtained from approximate representation of the plotted points with a straight line passing through an origin of the coordinate system, and

iii) performing division processing for dividing the data concerning the expression quantities with respect to the plurality of the genes, which expression quantities have been obtained for the second sample, by the value of the slope of the approximate straight line, whereby the data concerning the expression quantities having been obtained for the second sample are normalized.

In the first and second methods of normalizing gene expression data in accordance with the present invention, as each of the first and second samples, for example, a nucleic acid, such as a DNA or a genome, which has been extracted from a cell or a tissue, may be employed. The samples should preferably be set such that a sample obtained from a normal cell is employed as the first sample, and a sample obtained from an abnormal cell, e.g. a cell in a diseased state, is employed as the second sample. However, the first sample and the second sample are not limited to the samples described above. For example, a sample obtained from an abnormal cell may be employed as the first sample, and a sample obtained from a normal cell may be employed as the second sample. Alternatively, samples obtained from an abnormal cell may be employed as both the first and second samples.

With the first method of normalizing gene expression data in accordance with the present invention, in the process for comparing the expression quantities with respect to the plurality of the genes, which expression quantities have been obtained for the first sample, and the expression quantities with respect to the plurality of the genes, which expression quantities have been obtained for the second sample, with each other, the data concerning the expression quantities, which have been obtained for the first sample and the second sample, are indicated with the points plotted on the logarithmic coordinate system, in which the horizontal axis represents the logarithms of the expression quantities obtained for the first sample, and in which the vertical axis represents the logarithms of the expression quantities obtained for the second sample. Also, the coefficient is calculated from the value of the intercept of the approximate straight line, which is obtained from the approximate representation of the plotted points with the straight line having a slope of 1, on the vertical axis. Further, the division processing is performed, wherein the data concerning the expression quantities with respect to the plurality of the genes, which expression quantities have been obtained for the second sample, are divided by the coefficient. In this manner, the data concerning the expression quantities having been obtained for the second sample are normalized. Therefore, the problems are capable of being prevented from occurring in that, in cases where the expression quantity with respect to a certain gene, which is not necessarily representative of the expression quantities with respect to all of the genes, is taken as a reference expression quantity, the accuracy of the analysis becomes low. Also, the problems are capable of being prevented from occurring in that the normalized gene expression data are largely affected by the expression data with respect to genes having large expression quantities. Therefore, with the first method of normalizing gene expression data in accordance with the present invention, the gene expression data, which have been obtained for one of two samples with the micro array technique, and the gene expression data, which have been obtained for the other sample with the micro array technique, are capable of being compared accurately with each other and analyzed accurately. Accordingly, accurate judgments are capable of being made with respect to diagnosis and prevention of a disease, possibility of a person suffering from a disease, and the like. Also, the expression quantities with respect to the plurality of the genes, which expression quantities have been obtained for one of the two samples, and the expression quantities with respect to the plurality of the genes, which expression quantities have been obtained for the other sample, are capable of being simultaneously compared with each other with high accuracy. Therefore, in cases where a gene exhibiting different expression levels with respect to the two samples is found and in cases where, for example, the expression results having been obtained for a sample obtained from a person, who has suffered from a disease and has not been infected, and the expression results having been obtained for a sample obtained from a person, who has been infected, are compared with each other, a gene imparting resistance to the disease is capable of being identified. Further, comparison of expression levels may be made between tissue samples, which are at successive stages of an identical disease or at successive progress levels of an identical disease, or between tissue samples, which have been known to show different final results of a disease. In such cases, as for the cases of, for example, cancer, comparison of the expression levels between a malignant tissue and a benign tissue is capable of being made.

With the second method of normalizing gene expression data in accordance with the present invention, in the process for comparing the expression quantities with respect to the plurality of the genes, which expression quantities have been obtained for the first sample, and the expression quantities with respect to the plurality of the genes, which expression quantities have been obtained for the second sample, with each other, the data concerning the expression quantities, which have been obtained for the first sample and the second sample, are indicated with the points plotted on the coordinate system, in which the horizontal axis represents the expression quantities obtained for the first sample, and in which the vertical axis represents the expression quantities obtained for the second sample. Also, the value of the slope of the approximate straight line, which is obtained from approximate representation of the plotted points with the straight line passing through the origin of the coordinate system, is calculated. Further, the division processing is performed, wherein the data concerning the expression quantities with respect to the plurality of the genes, which expression quantities have been obtained for the second sample, are divided by the value of the slope of the approximate straight line. In this manner, the data concerning the expression quantities having been obtained for the second sample are normalized. Therefore, with the second method of normalizing gene expression data in accordance with the present invention, the same effects as those described above are capable of being obtained.

### BRIEF DESCRIPTION OF THE DRAWINGS

FIG. 1 is a graph showing logarithms of expression quantities with respect to a plurality of genes, which expression quantities have been obtained for a first sample in a first embodiment of the method of normalizing gene expression data in accordance with the present invention, and the logarithms of the expression quantities with respect to the plurality of the genes, which expression quantities have been obtained for a second sample in the first embodiment of the method of normalizing gene expression data in accordance with the present invention,

FIG. 2 is a graph obtained from correction of the graph shown in FIG. 1,

FIG. 3 is a graph showing logarithms of expression quantities with respect to a plurality of genes, which expression quantities have been obtained for a first sample in a second embodiment of the method of normalizing gene expression data in accordance with the present invention, and the logarithms of the expression quantities with respect to the plurality of the genes, which expression quantities have been obtained for a second sample in the second embodiment of the method of normalizing gene expression data in accordance with the present invention, and

FIG. 4 is a graph obtained from correction of the graph shown in FIG. 3.

### DESCRIPTION OF THE PREFERRED EMBODIMENTS

The present invention will hereinbelow be described in further detail with reference to the accompanying drawings.

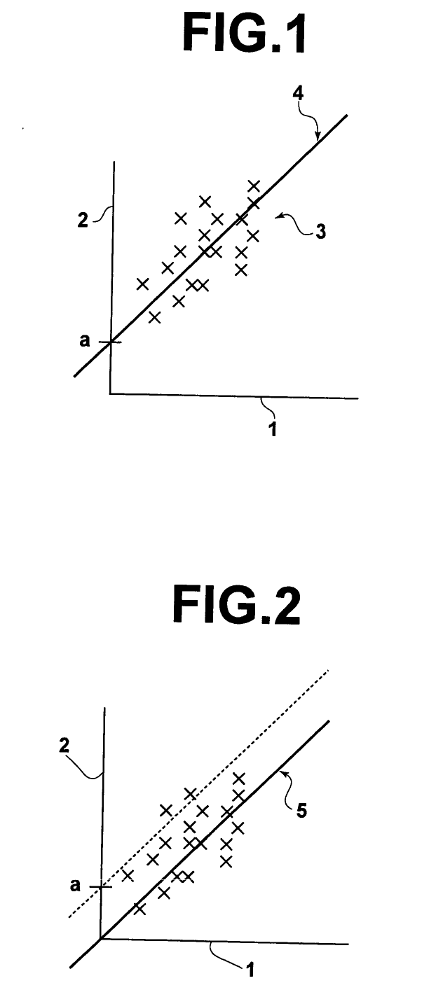

FIG. 1 is a graph showing logarithms of expression quantities with respect to a plurality of genes, which expression quantities have been obtained for a first sample in a first embodiment of the method of normalizing gene expression data in accordance with the present invention, and the logarithms of the expression quantities with respect to the plurality of the genes, which expression quantities have been obtained for a second sample in the first embodiment of the method of normalizing gene expression data in accordance with the present invention. In FIG. 1, a horizontal axis 1 represents the logarithms of the gene expression quantities obtained for the first sample, and a vertical axis 2 represents the logarithms of the gene expression quantities obtained for the second sample. With respect to a gene, whose expression has been measured for both the first sample and the second sample with the micro array technique, the gene expression quantity obtained for the first sample may be measured as being x, and the gene expression quantity obtained for the second sample may be measured as being y. In such cases, the logarithm, log x, of the gene expression quantity obtained for the first sample and the logarithm, log y, of the gene expression quantity obtained for the second sample are indicated with plotted points 3, 3, . . . on the logarithmic coordinate system having the horizontal axis 1 and the vertical axis 2.

In FIG. 1, an approximate straight line 4, which is obtained from approximate representation of the plurality of the plotted points 3, 3, . . . with a straight line having a slope of 1, is drawn. Also, a value of an intercept of the approximate straight line 4 on the vertical axis 2 is found as being “a.” In such cases, the approximate straight line 4 may be represented by Formula (1) shown below.

log y=log x+a (1)

In order for the gene expression data, which have been obtained for the first sample, and the gene expression data, which have been obtained for the second sample, to be appropriately compared with each other, the approximate straight line 4 shown in FIG. 1 should preferably be corrected to the straight line shown in FIG. 2, which straight line passes through the origin of the logarithmic coordinate system. The corrected straight line may be represented by Formula (2) shown below.

log y′=log x (2)

In Formula (2) shown above, log y′=log x−a.

Therefore, Formula (3) shown below obtains.

y′=y/10^{a} (3)

Accordingly, in cases where division processing is performed, wherein the data concerning the expression quantities with respect to the plurality of the genes, which expression quantities have been obtained for the second sample, are divided by the coefficient, 10^{a}, the data concerning the expression quantities having been obtained for the second sample are capable of being normalized appropriately. The data concerning the expression quantities with respect to the plurality of the genes, which expression quantities have been obtained for the first sample, and the normalized data concerning the expression quantities with respect to the plurality of the genes, which expression quantities have been obtained for the second sample, may then be compared with each other. In this manner, accurate judgments are capable of being made with respect to diagnosis and prevention of a disease, possibility of a person suffering from a disease, and the like.

In the first embodiment of the method of normalizing gene expression data in accordance with the present invention, the plotted points 3, 3, . . . are approximately represented by the straight line having a slope of 1. Alternatively, the plotted points 3, 3, . . . may be approximately represented by a straight line having a slope other than 1. As another alternative, the plotted points 3, 3, . . . may be approximately represented by a curved line, or the like.

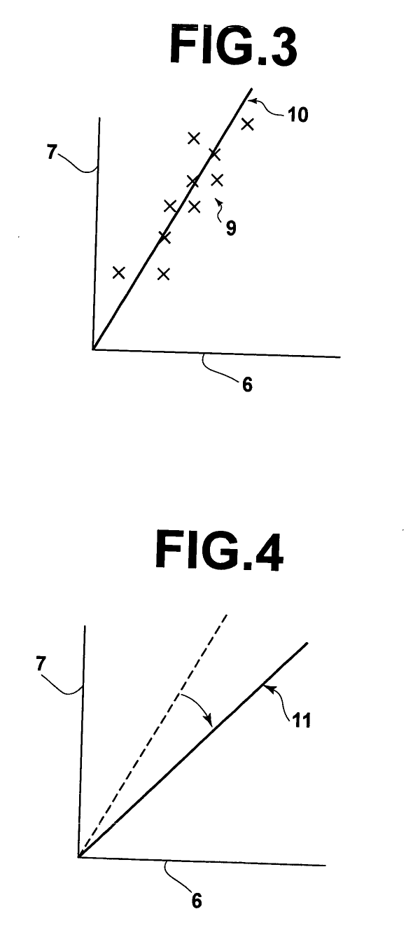

FIG. 3 is a graph showing logarithms of expression quantities with respect to a plurality of genes, which expression quantities have been obtained for a first sample in a second embodiment of the method of normalizing gene expression data in accordance with the present invention, and the logarithms of the expression quantities with respect to the plurality of the genes, which expression quantities have been obtained for a second sample in the second embodiment of the method of normalizing gene expression data in accordance with the present invention. In FIG. 3, a horizontal axis 6 represents the gene expression quantities obtained for the first sample, and a vertical axis 7 represents the gene expression quantities obtained for the second sample. With respect to a gene, whose expression has been measured for both the first sample and the second sample with the micro array technique, the gene expression quantity obtained for the first sample may be measured as being x, and the gene expression quantity obtained for the second sample may be measured as being y. In such cases, the gene expression quantity obtained for the first sample and the gene expression quantity obtained for the second sample are indicated with plotted points 9, 9, . . . on the coordinate system having the horizontal axis 6 and the vertical axis 7.

In FIG. 3, an approximate straight line 10, which is obtained from approximate representation of the plurality of the plotted points 9, 9, . . . with a straight line passing through the origin of the coordinate system, is drawn. Also, a value of a slope of the approximate straight line 10 is found as being “b.” In such cases, the approximate straight line 10 may be represented by Formula (4) shown below.

y=bx (4)

In order for the gene expression data, which have been obtained for the first sample, and the gene expression data, which have been obtained for the second sample, to be appropriately compared with each other, the approximate straight line 10 shown in FIG. 3 should preferably be corrected to a straight line 11 shown in FIG. 4, which straight line passes through the origin of the coordinate system and has a slope of 1. The corrected straight line 11 may be represented by Formula (5) shown below.

y′=x (5)

In Formula (5) shown above, Formula (6) shown below obtains.

y′=y/b (6)

Therefore, in cases where division processing is performed, wherein the data concerning the expression quantities with respect to the plurality of the genes, which expression quantities have been obtained for the second sample, are divided by the coefficient “b,” the data concerning the expression quantities having been obtained for the second sample are capable of being normalized appropriately. Accordingly, the same effects as those described above are capable of being obtained.

In FIG. 1 and FIG. 3, in cases where variation occurs with the distribution of the plotted points, it may occur that an appropriate approximate straight line cannot be drawn due to adverse effects of densely distributed plotted points. In such cases, the region of the coordinate system may be divided into several blocks, and the plotted points may be selected at random in each of the blocks such that the number of the plotted points may be identical in every block. In such cases, an approximate straight line may be formed by use of the plotted points having been selected. In this manner, the approximate straight line is capable of being formed with all of the data concerning large expression quantities and the data concerning small expression quantities being taken into consideration.

Also, in the embodiments described above, the gene expression data having been obtained for the second sample are normalized. Alternatively, the gene expression data having been obtained for the first sample may be normalized. As another alternative, both the gene expression data having been obtained for the first sample and the gene expression data having been obtained for the second sample may be normalized.

What is claimed is:

1. A method of normalizing gene expression data wherein, in a process for comparing expression quantities with respect to a plurality of genes, which expression quantities have been obtained for a first sample, and expression quantities with respect to the plurality of the genes, which expression quantities have been obtained for a second sample, with each other, data concerning the expression quantities having been obtained for the second sample are normalized, the method comprising the steps of:
i) indicating the data concerning the expression quantities, which have been obtained for the first sample and the second sample, with points plotted on a logarithmic coordinate system, in which a horizontal axis represents logarithms of the expression quantities obtained for the first sample, and in which a vertical axis represents logarithms of the expression quantities obtained for the second sample,
ii) calculating a coefficient from a value of an intercept of an approximate straight line, which is obtained from approximate representation of the plotted points with a straight line having a slope of 1, on the vertical axis, and
iii) performing division processing for dividing the data concerning the expression quantities with respect to the plurality of the genes, which expression quantities have been obtained for the second sample, by the coefficient, whereby the data concerning the expression quantities having been obtained for the second sample are normalized.

2. A method as defined in claim 1 wherein the first sample is a sample obtained from a normal cell, and the second sample is a sample obtained from an abnormal cell.

3. A method of normalizing gene expression data wherein, in a process for comparing expression quantities with respect to a plurality of genes, which expression quantities have been obtained for a first sample, and expression quantities with respect to the plurality of the genes, which expression quantities have been obtained for a second sample, with each other, data concerning the expression quantities having been obtained for the second sample are normalized, the method comprising the steps of:
i) indicating the data concerning the expression quantities, which have been obtained for the first sample and the second sample, with points plotted on a coordinate system, in which a horizontal axis represents the expression quantities obtained for the first sample, and in which a vertical axis represents the expression quantities obtained for the second sample,
ii) calculating a value of a slope of an approximate straight line, which is obtained from approximate representation of the plotted points with a straight line passing through an origin of the coordinate system, and
iii) performing division processing for dividing the data concerning the expression quantities with respect to the plurality of the genes, which expression quantities have been obtained for the second sample, by the value of the slope of the approximate straight line, whereby the data concerning the expression quantities having been obtained for the second sample are normalized.

4. A method as defined in claim 3 wherein the first sample is a sample obtained from a normal cell, and the second sample is a sample obtained from an abnormal cell.
